(19) United States
(12) Patent Application Publication
(10) Pub. No.: US 2024/0065704 A1
(43) Pub. Date: Feb. 29, 2024

(54) ENDOSCOPIC SURGICAL CLIP APPLIER

(71) Applicant: Covidien LP, Mansfield, MA (US)

(72) Inventors: Roy J. Pilletere, Middletown, CT (US); Eric Brown, Madison, CT (US); Jacob C. Baril, Norwalk, CT (US); Gregory R. Morck, Haddam, CT (US); Saumya Banerjee, Southington, CT (US); Justin Thomas, New Haven, CT (US); Matthew A. Dinino, Newington, CT (US)

(21) Appl. No.: 18/504,498

(22) Filed: Nov. 8, 2023

Related U.S. Application Data
(63) Continuation of application No. 17/474,673, filed on Sep. 14, 2021, now Pat. No. 11,812,972.
Application No. 17/474,673 is a continuation of application No. 16/532,551, filed on Aug. 6, 2019, now Pat. No. 11,147,566.
(60) Provisional application No. 62/739,421, filed on Oct. 1, 2018.

(51) Int. Cl. A61B 17/128

(52) U.S. Cl. CPC A61B 17/1285

(57) ABSTRACT

A surgical clip applier includes a handle assembly, an elongated shaft, a drive shaft, a cam pin, and an end effector. The cam pin is disposed in mechanical cooperation with the drive shaft. The end effector is disposed adjacent a distal end of the elongated shaft and includes a first jaw member and a second jaw member. The end effector is disposed in operative engagement with the drive shaft such that longitudinal translation of the drive shaft relative to the housing of the handle assembly causes the first jaw member to move toward the second jaw member. The first jaw member includes a first cam slot configured to slidingly receive the cam pin. The first cam slot defines a first portion having a first slope and a second portion having second slope.

### CROSS-REFERENCE TO RELATED APPLICATIONS

This application is a continuation application of U.S. patent application Ser. No. 17/474,673, filed on Sep. 14, 2021, which is a continuation application of U.S. patent application Ser. No. 16/532,551, filed on Aug. 6, 2019 (now U.S. Pat. No. 11,147,566), which claims the benefit of and priority to U.S. Provisional Patent Application No. 62/739,421 filed on Oct. 1, 2018, the entire contents of each of which are incorporated by reference herein.

### BACKGROUND

This disclosure relates to surgical clip appliers. More particularly, the present disclosure relates to endoscopic surgical clip appliers having jaw members with cam slots to optimize use.

Endoscopic surgical staplers and surgical clip appliers are known in the art and are used for a number of distinct and useful surgical procedures. In the case of a laparoscopic surgical procedure, access to the interior of an abdomen is achieved through narrow tubes or cannulas inserted through a small entrance incision in the skin. Minimally invasive procedures performed elsewhere in the body are often generally referred to as endoscopic procedures. Typically, a tube or cannula device is extended into the patient's body through the entrance incision to provide an access port. The port allows the surgeon to insert a number of different surgical instruments therethrough using a trocar and for performing surgical procedures far removed from the incision.

During a majority of these procedures, the surgeon must often terminate the flow of blood or another fluid through one or more vessels. The surgeon will often use a particular endoscopic surgical clip applier to apply a surgical clip to a blood vessel or another duct to prevent the flow of body fluids therethrough during the procedure.

Endoscopic surgical clip appliers having various sizes (e.g., diameters), that are configured to apply a variety of diverse surgical clips, are known in the art, and which are capable of applying a single or multiple surgical clips during an entry to the body cavity. Such surgical clips are typically fabricated from a biocompatible material and are usually compressed over a vessel. Once applied to the vessel, the compressed surgical clip terminates the flow of fluid therethrough.

To apply the surgical clip to the vessel, an appropriate amount of force is typically applied to jaw members of the endoscopic surgical clip appliers, which hold the surgical clip therebetween. The applied force causes at least one jaw member to move toward the other and thereby compress the surgical clip. As the force may be applied by a physician using a pivotable handle of the endoscopic surgical clip applier, for example, reducing the amount of force required to properly form the surgical clip may be advantageous. Additionally, following the formation of the surgical clip, the jaw members are typically removed from the surgical site through the trocar. Minimizing the force required to remove the jaw members through the trocar (e.g., by pivoting at least one jaw member toward the other) may also be advantageous.

### SUMMARY

The present disclosure relates to a surgical clip applier including a handle assembly, an elongated shaft, a drive shaft, a cam pin, and an end effector. The handle assembly includes a housing and a trigger pivotally connected to the housing. The elongated shaft extends distally from the handle assembly. The drive shaft is mechanically engaged with the trigger and is longitudinally translatable relative to the housing of the handle assembly in response to actuation of the trigger. The cam pin is disposed in mechanical cooperation with the drive shaft. The end effector is disposed adjacent a distal end of the elongated shaft and includes a first jaw member and a second jaw member. The end effector is disposed in operative engagement with the drive shaft such that longitudinal translation of the drive shaft relative to the housing of the handle assembly causes the first jaw member to move toward the second jaw member. The first jaw member includes a first cam slot configured to slidingly receive the cam pin. The first cam slot defines a first portion having a first slope relative to a longitudinal axis of the first jaw member and a second portion having second slope relative to the longitudinal axis of the first jaw member.

In disclosed embodiments, the first portion of the cam slot is disposed proximally of the second portion of the cam slot, and the first slope defines an angle relative to the longitudinal axis of the first jaw member that is steeper than the second slope relative to the longitudinal axis of the first jaw member. It is also disclosed that the first portion of the cam slot defines a shorter length than a length of the second portion of the cam slot.

Further, it is disclosed that the angle defined by the first slope is between about 35° and about 45°, and the angle defined by the second slope is between about 15° and about 20°.

It is additionally disclosed that the first portion of the cam slot defines a length of between about 0.075 inches and about 0.125 inches, and the second portion of the cam slot defines a length of between about 0.175 inches and about 0.225 inches.

It is also disclosed that the second jaw member includes a second cam slot configured to slidingly receive the cam pin. In embodiments, the second cam slot defines a first portion having a first slope relative to a longitudinal axis of the second jaw member and a second portion having second slope relative to the longitudinal axis of the second jaw member. It is further disclosed that the second cam slot of the second jaw member substantially mirrors the first cam slot of the first jaw member.

In disclosed embodiments, the cam slot defines a transition area interconnecting the first portion of the cam slot and the second portion of the cam slot.

### BRIEF DESCRIPTION OF THE DRAWINGS

A particular embodiment of a surgical clip applier is disclosed herein with reference to the drawings wherein:

FIG. 1 is a perspective view of a surgical clip applier in accordance with an embodiment of the present disclosure;

FIG. 2 is a perspective view, with parts separated, of an endoscopic assembly of the surgical clip applier of FIG. 1;

FIG. 3 is a top, plan view of the endoscopic assembly of FIGS. 1 and 2;

FIG. 4 is a transverse, cross-sectional view of the endoscopic assembly of FIGS. 1-3, as taken through 4-4 of FIG. 3;

FIG. 5 is a side view of a jaw member of the surgical clip applier of FIGS. 1-4;

FIGS. 6-8 are side views of jaw members of the surgical clip applier of FIGS. 1-4 at different stages of operation and including a surgical clip supported therebetween; and

FIG. 9 is a schematic illustration of a robotic surgical system configured for use in accordance with the present disclosure.

### DETAILED DESCRIPTION

Embodiments of endoscopic surgical clip appliers, in accordance with the present disclosure, will now be described in detail with reference to the drawing figures wherein like reference numerals identify similar or identical structural elements. As shown in the drawings and described throughout the following description, as is traditional when referring to relative positioning on a surgical instrument, the term “proximal” refers to the end of the apparatus which is closer to the user and the term “distal” refers to the end of the apparatus which is farther away from the user.

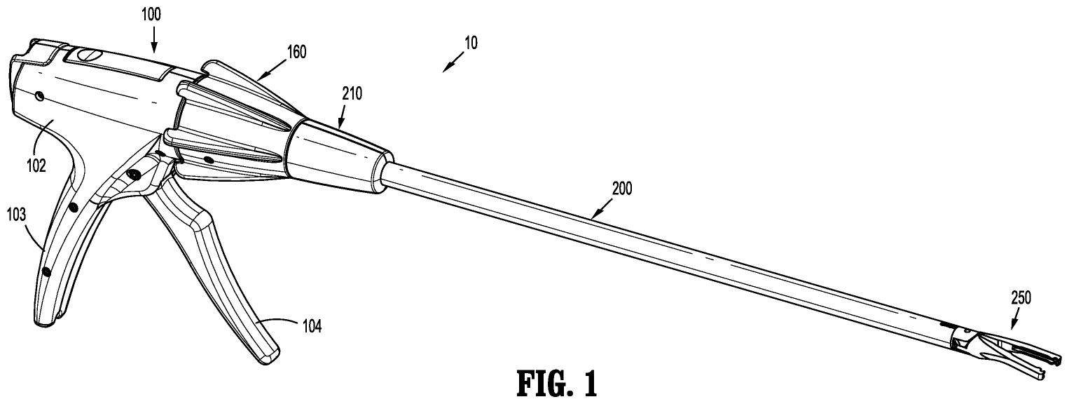

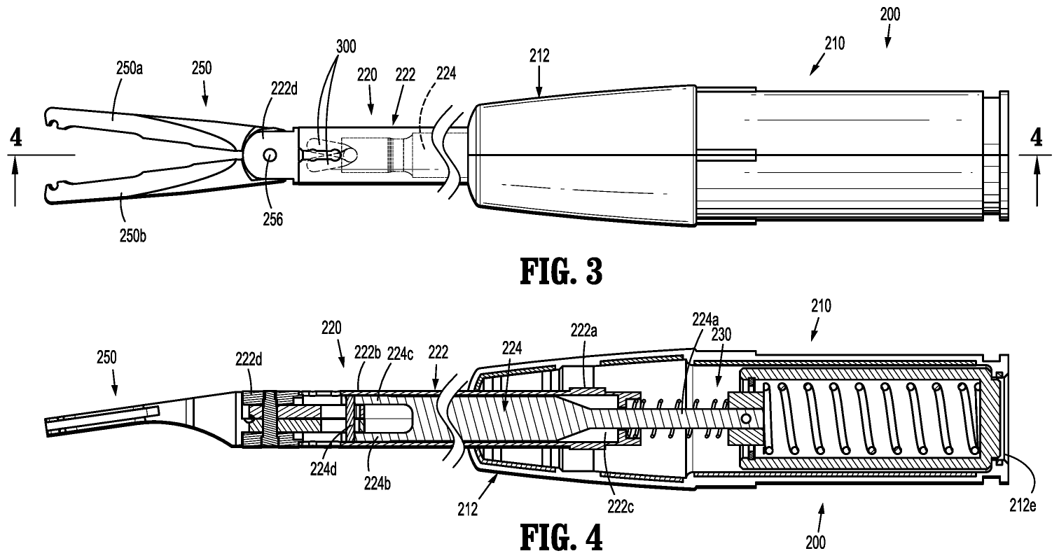

Referring now to FIGS. 1-8, an endoscopic surgical clip applier in accordance with an embodiment of the present disclosure, and is generally designated as reference number 10. Surgical clip applier 10 generally includes a handle assembly or actuation assembly 100, an endoscopic assembly 200 extending distally from handle assembly 100, a pair of jaw members 250 extending distally from endoscopic assembly 200, and optionally, at least one disposable surgical clip cartridge assembly (not shown) selectively loadable into a shaft assembly of endoscopic assembly 200.

Briefly, the shaft assembly of endoscopic assembly 200 may have various outer diameters such as, for example, about 5 mm or about 10 mm, depending on intended use. Further, the shaft assembly may have various relatively elongated or shortened lengths depending on intended use, such as, for example, in bariatric surgery. In one embodiment, in bariatric surgery, the shaft assembly may have a length of between about 30 cm and about 40 cm. Further, the shaft assembly may be configured to fire and form a specific type of surgical clip, either individually or multiply. However one skilled in the art should appreciate that the shaft assembly may have any length in excess of about 30 cm and the present disclosure is not limited to any of the above identified lengths.

In accordance with the present disclosure, endoscopic assembly 200 or a surgical clip cartridge assembly (not shown) may be loaded with a particularly sized set of surgical clips 500 (e.g., relatively small surgical clips, relatively medium surgical clips, or relatively large surgical clips), an example of which is shown in FIGS. 6-8. It is contemplated that clip cartridge assemblies may be configured to be selectively loaded into the shaft assembly of endoscopic assembly 200, and to be actuated by the same or common handle assembly 100 to fire and form the surgical clip(s) 500 loaded therein onto underlying tissue and/or vessels.

Referring now to FIG. 1, handle assembly 100 of surgical clip applier 10 is shown and will be described. Generally, handle assembly 100 includes a housing 102, a stationary handle 103, a trigger 104, a drive plunger (not shown), and a rotation knob 160. Drive plunger is operatively connected to trigger 104 and is slidably supported within housing 102 of handle assembly 100. Actuation of trigger 104 toward stationary handle 103 distally advances the drive plunger relative to housing 102. Rotation knob 160 is disposed at a distal portion of housing 102 and enables endoscopic assembly 200 to rotate 360° about a longitudinal axis thereof relative to housing 102 of handle assembly 100.

Further details of endoscopic surgical clip appliers are described in U.S. patent application Ser. No. 15/341,292, filed on Nov. 2, 2016 (now U.S. Pat. No. 10,390,831), the entire contents of which is incorporated herein by reference.

Turning now to FIGS. 1-4, endoscopic assembly 200 of surgical clip applier 10 is shown and described. Endoscopic assembly 200 includes a hub assembly 210, a shaft assembly or elongated shaft 220 extending from hub assembly 210, and a pair of jaw members 250 pivotally connected to a distal end of shaft assembly 220. It is contemplated that endoscopic assembly 200 may be configured to close, fire or form surgical clips 500 similar to those shown and described in U.S. Pat. No. 4,834,096, the entire contents of which is incorporated herein by reference.

An outer housing 212 of hub assembly 210 further defines an open proximal end 212e configured to slidably receive a distal end of the drive plunger of handle assembly 100, when endoscopic assembly 200 is coupled to handle assembly 100 and/or when surgical clip applier 10 is fired.

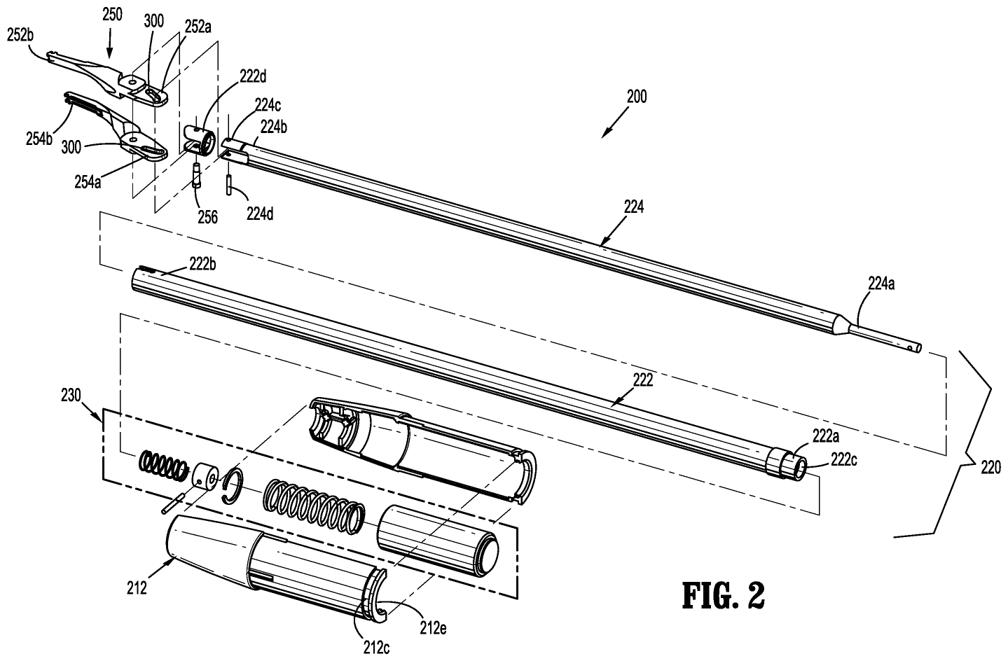

Shaft assembly 220 of endoscopic assembly 200 includes an elongate outer tube 222 having a proximal end 222a supported and secured to outer housing 212 of hub assembly 210, a distal end 222b projecting from outer housing 212 of hub assembly 210, and a lumen 222c (FIGS. 2 and 4) extending longitudinally therethrough. As shown in FIGS. 2 and 4, hub assembly 210 includes a drive assembly 230 supported within outer housing 212 thereof. Distal end 222b of outer tube 222 supports or defines an outer clevis 222d for pivotally supporting a pair of jaw members 250, as will be described in greater detail below.

Shaft assembly 220 further includes an inner shaft or drive shaft 224 slidably supported within lumen 222c of outer tube 222. Inner shaft 224 includes a proximal end 224a projecting proximally from proximal end 222a of outer tube 222, and a distal end 224b defining an inner clevis 224c for supporting a cam pin 224d (FIGS. 2 and 4) which engages camming slots 300 of jaw members 250, as will be described in greater detail below.

As illustrated in FIGS. 2 and 4, endoscopic assembly 200 includes a pair of jaw members 250 pivotally supported in a clevis 222d at distal end 222b of outer tube 222 by a pivot pin 256 extending through a pivot hole 251 of each jaw member 250. The pair of jaw members 250 includes a first jaw 250a and a second jaw 250b. Each jaw member 250a, 250b includes a respective proximal end 252a, 252b, and a respective distal end 254a, 254b, wherein proximal ends 252a, 252b and distal ends 254a, 254b of jaw members 250a, 250b are pivotable about pivot pin 256. Each proximal end 252a, 252b of respective jaw members 250a, 250b defines a cam slot 300a, 300b therein which is sized and configured to receive cam pin 224d of inner shaft 224. In use, as inner shaft 224 is axially displaced relative to outer shaft 222, inner shaft 224 translates cam pin 224d thereof through cam slots 300a, 300b of respective jaw members 250a, 250b, to thereby open or close jaw members 250.

When the pair of j aw members 250 is in an open position, and a new, unformed or open surgical clip 500 is located or loaded within the distal ends 254a, 254b of jaw members 250a, 250b, and as inner shaft 224 is moved distally relative to outer shaft 222, cam pin 224d is translated through cam slots 300a, 300b of respective jaw members 250a, 250b. As cam pin 224d is translated through cam slots 300a, 300b of jaw members 250a, 250b, the distal ends 254a, 254b of jaw members 250a, 250b are moved toward the closed or approximated position to close and/or form the surgical clip 500 located or loaded therewithin.

The dimensions of jaw members 250a, 250b and cam slots 300a, 300b determine an overall length required to move jaw members 250a, 250b from a fully open position (FIG. 6) to a fully closed position (FIG. 8), defining a closure stroke length of the pair of jaw members 250.

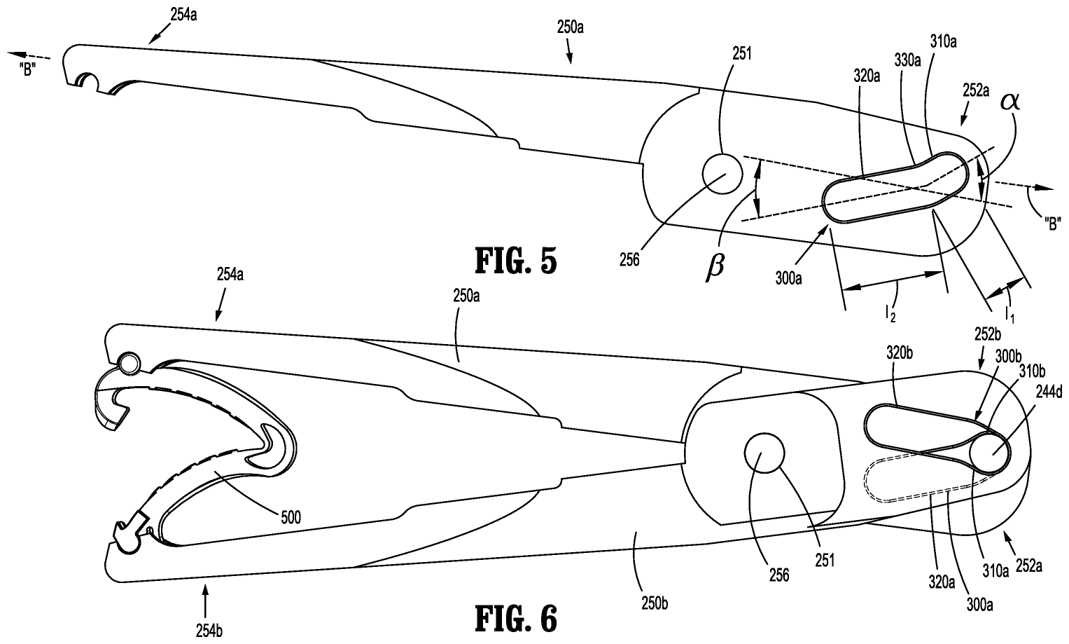

With particular reference to FIGS. 5-8, further details of cam slots 300 are described. Cam slots 300 are configured to both minimize closure forces associated with approximating jaw members 250 for properly forming surgical clips 500, and to minimize the forces associated with removing jaw members 250 from a surgical site through a trocar. In general, a cam slot having a relatively steep angle requires greater force (relative to a shallower angle) from inner shaft 224 (and thus trigger 104) in order to make cam pin 224d travel therethrough. However, following the formation of surgical clips 500, a relatively steep angle of the cam slot makes removal of surgical clip applier 10 from the trocar easier than a shallower angle of the cam slot, as less removal force is required. Thus, either minimizing the force required to close jaw members or minimizing force required to remove jaw members of a surgical clip applier through a trocar is typically sacrificed when designing jaw members 250.

With continued reference to FIGS. 5-8, jaw members 250 and cam slots 300 of the present disclosure are configured to both minimize the force required to close jaw members 250, and to minimize the force associated with removing surgical clip applier 10 through a trocar (e.g., the force required to pull surgical clip applier 10 proximally to approximate the jaw members 250 a sufficient amount such that jaw members 250 fit within the trocar). That is, when removing surgical clip applier 10 from the trocar, the inner diameter of the trocar acts upon outer surfaces of jaw members 250, against a bias (e.g., a spring-loaded bias which urges jaw members 250 open). The steeper angle of cam slots 300, as discussed below, allows cam pin 224d to slide smoother and/or easier relative to a shallower angle. Thus, less force is required to close jaw members 250 when pushing them closed from their outer surfaces.

With particular reference to FIG. 5, a single jaw member 250a is shown. It is envisioned that the other jaw member 250b is a mirror image or a substantially mirror image as jaw member 250a. As shown, cam slot 300a includes a first portion 310a having a first slope, and a second portion 320a having a second slope. First portion 310a defines a linear path through which cam pin 224d translates, and second portion 320a defines a linear path through which cam pin 224d translates. As shown, first portion 310a defines a first angle α with respect to an axis “B” of jaw member 250a that is between about 35° and about 45°, and may be equal to about 40°. Second portion 320a defines a second angle β with respect to the axis “B” of jaw member 250 that is between about 15° and about 20°, and may be equal to about 18°. Thus, first portion 310a defines a greater or steeper angle than second portion 320a. While particular values for angle α and angle β are specified, other values are also contemplated by the present disclosure. Additionally, second portion 320a of slot 300a includes a longer length l₂, along which cam pin 224d translates, than a length l₁ of first portion 310a of slot 300a. It is envisioned that the length l₁ of first portion 310a of slot 300 is between about 0.075 inches and about 0.125 inches, and may be equal to about 0.100 inches, and that the length l₂ of second portion 320a of slot 300 is between about 0.175 inches and about 0.225 inches, and may be equal to about 0.200 inches.

Further, slot 300a defines a transition zone, point or area 330a interconnecting first portion 310a and second portion 320a of slot 300a.

Referring now to FIGS. 6-8, the movement of jaw members 250 is illustrated. FIG. 6 illustrates jaw members 250a and 250b in their open position with surgical clip 500 disposed therebetween, and prior to the compression or formation of surgical clip 500. In this position, cam pin 244d is located at a proximal end of first portion 310a, 310b of respective cam slots 300a, 300b.

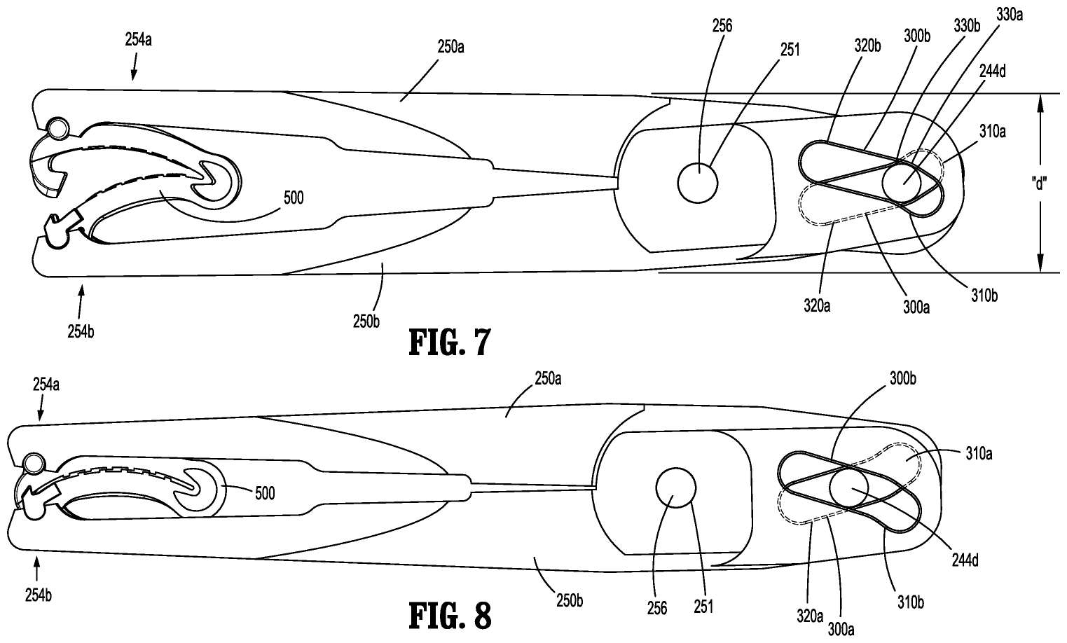

In FIG. 7, jaw members 250a and 250b are shown in a partially approximated position with surgical clip 500 having been somewhat compressed. In this position, cam pin 244d is located at transition area 330a, 330b between first portion 310a and second portion 320a of cam slot 300a, and between first portion 310b and second portion 320b of cam slot 300b. Thus, during initial and partial approximation of jaw members 250a, 250b, cam pin 244d moved along first portions 310a, 310b of respective cam slots 300a, 300b, which, as discussed above, include a relatively steep slope. While a relatively large amount of force is required to move cam pin 244d along the steep slope, it often requires less force to initially compress a surgical clip 500 than to fully compress the surgical clip 500. Accordingly, the initial steep slope of cam slots 300a, 300b paired with the relatively small amount of force required to initially compress a surgical clip 500 may not be too burdensome to the user of surgical clip applier 10.

With reference to FIG. 8, jaw members 250a and 250b are shown in their approximated position with surgical clip 500 having been fully compressed or formed. Here, cam pin 244d is located within second portion 320a, 320b of respective cam slots 300a, 300b. Thus, during the continued approximation of jaw members 250a, 250b (from the position shown in FIG. 7), cam pin 244d moves along second portions 320a, 320b of respective cam slots 300a, 300b, which, as discussed above, includes a relatively shallow slope. Due to the relatively shallow slop, advancing cam pin 244d through second portions 320a, 320b of cam slots 300a, 300b requires a relative small amount of force. Further, the compression of surgical clips 500 between a partially compressed configuration (FIG. 7) to a fully compressed configuration (FIG. 8) often requires more force than the initial compression of surgical clips 500 (from the configuration shown in FIG. 6 to the configuration shown in FIG. 7). Thus, the shallower slope during this stage of formation of surgical clip 500 mitigates the physical burden of actuation of trigger 106.

After the surgical clip 500 is formed and applied to tissue, for instance, jaw members 250a, 250b move to their original position (FIG. 6), in response to the biasing of jaw members 250a, 250b. Here, to remove surgical clip applier 10 from the trocar, jaw members 250a, 250b must be at least partially approximated such that the distance “d” (FIG. 7) between outer edges of jaw members 250a, 250b is smaller than an inner diameter of the trocar. In use, surgeons may not physically close jaw members 250 prior to removing surgical clip applier 10 from the trocar. In such situations, when removing surgical clip applier 10 from the trocar, the inner diameter of the trocar acts upon outer surfaces of jaw members 250 to approximate jaw members 250. Here, the steeper angle of first portions 310a, 310b of respective cam slots 300a, 300b allows cam pin 224d to slide with less force relative to a shallower angle. Thus, less force is required to close jaw members 250 when pushing them toward the approximated position from their outer surfaces.

To the extent consistent, handle assembly 100 and/or endoscopic assembly 200 may include any or all of the features of the handle assembly and/or endoscopic assemblies disclosed and described in International Patent Application No. PCT/CN2015/080845, filed Jun. 5, 2015, entitled “Endoscopic Reposable Surgical Clip Applier,” International Patent Application No. PCT/CN2015/091603, filed on Oct. 10, 2015, entitled “Endoscopic Surgical Clip Applier,” International Patent Application No. PCT/CN2015/093626, filed on Nov. 3, 2015, entitled “Endoscopic Surgical Clip Applier,” and/or PCT/CN2015/094195, filed on Nov. 10, 2015, entitled “Endoscopic Reposable Surgical Clip Applier,” the entire contents of each of which being incorporated herein by reference.

Surgical instruments such as the clip appliers described herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

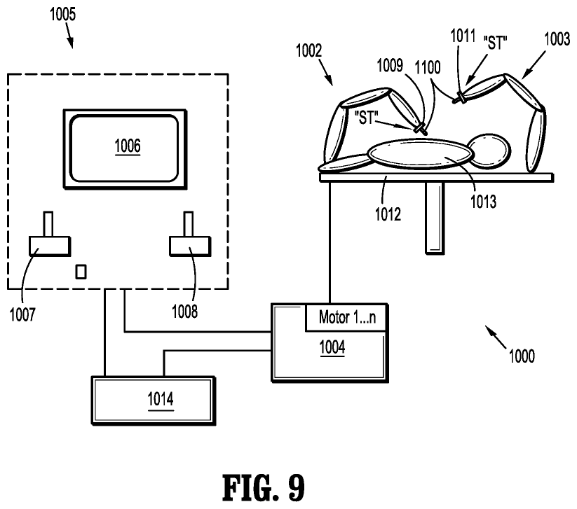

Referring to FIG. 9, a medical work station is shown generally as work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus the surgical tool (including end effector 1100) execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of end effector 1100. Medical work station 1000 may also include more than two robot arms 1002, 1003, the additional robot arms likewise being connected to control device 1004 and being telemanipulatable by means of operating console 1005. A medical instrument or surgical tool (including an end effector 1100) may also be attached to the additional robot arm. Medical work station 1000 may include a database 1014, in particular coupled to with control device 1004, in which are stored, for example, pre-operative data from patient/living being 1013 and/or anatomical atlases.

Reference is made herein to U.S. Patent Publication No. 2012/0116416, filed on Nov. 3, 2011 (now U.S. Pat. No. 8,828,023), entitled “Medical Workstation,” the entire contents of which is incorporated herein by reference, for a more detailed discussion of the construction and operation of an exemplary robotic surgical system.

It is contemplated, and within the scope of the present disclosure, that other endoscopic assemblies, including a pair of jaw members having a unique and diverse closure stroke length thereof, may be provided with a drive assembly, similar to any of the drive assemblies described herein, for accommodating and adapting the closure stroke length for the pair of jaw members thereof to the constant trigger stroke length.

Accordingly, various endoscopic assemblies, constructed in accordance with the principles of the present disclosure, may be provided which are also capable of firing or forming or closing surgical clips of various sizes, materials, and configurations, across multiple platforms for multiple different manufactures. For example, while the configuration of jaw members 250a and 250b have been shown and described for use with a surgical clip applier, it is contemplated and within the scope of the present disclosure that the configuration of jaw members 250a, 250b may be incorporated into other surgical instruments, such as, for example, and not limited to, surgical staplers, surgical graspers, surgical dissectors, and the like.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

1. (canceled)

2. A method of approximating jaw members of a surgical instrument, comprising:
moving a pin through a first portion of a cam slot of a first jaw member to cause initial movement of the first jaw member relative to a second jaw member, the first portion of the cam slot having a first slope relative to a longitudinal axis defined by the first jaw member; and
moving the pin through a second portion of the cam slot of the first jaw member to cause additional movement of the first jaw member relative to the second jaw member, the second portion of the cam slot having a second slope relative to the longitudinal axis, the second slope being different than the first slope.

3. The method according to claim 2, wherein moving the pin through the first portion of the cam slot occurs prior to moving the pin through the second portion of the cam slot.

4. The method according to claim 2, wherein moving the pin through the first portion of the cam slot includes moving the pin distally relative to the first jaw member.

5. The method according to claim 4, wherein moving the pin through the second portion of the cam slot includes moving the pin distally relative to the first jaw member.

6. The method according to claim 5, wherein moving the pin through the first portion of the cam slot occurs prior to moving the pin through the second portion of the cam slot.

7. The method according to claim 2, wherein moving the pin through the first portion of the cam slot of the first jaw member includes moving the pin a first distance, and moving the pin through the second portion of the cam slot of the first jaw member includes moving the pin a second distance, the first distance being different than the second distance.

8. The method according to claim 7, wherein the first distance is smaller than the second distance.

9. The method according to claim 8, wherein the second slope of the cam slot defines a steeper angle relative to the longitudinal axis than the first slope of the cam slot.

10. The method according to claim 2, wherein the second slope of the cam slot defines a steeper angle relative to the longitudinal axis than the first slope of the cam slot.

11. The method according to claim 2, further including moving the pin through a first portion of a cam slot of the second jaw member, and moving the pin through a second portion of the cam slot of the second jaw member.

12. The method according to claim 11, wherein moving the pin through the first portion of the cam slot of the first jaw member occurs at the same time as moving the pin through the first portion of the cam slot of the second jaw member.

13. The method according to claim 12, wherein moving the pin through the second portion of the cam slot of the first jaw member occurs at the same time as moving the pin through the second portion of the cam slot of the second jaw member.

14. A method of compressing a surgical clip, the method comprising:
moving a pin through a first portion of a cam slot of a first jaw member to cause a partial compression of a surgical clip positioned between the first jaw member and a second jaw member, the first portion of the cam slot having a first slope relative to a longitudinal axis defined by the first jaw member; and
moving the pin through a second portion of the cam slot of the first jaw member to cause a subsequent compression of the surgical clip positioned between the first jaw member and the second jaw member, the second portion of the cam slot having a second slope relative to the longitudinal axis, the second slope being different than the first slope.

15. The method according to claim 14, wherein moving the pin through the first portion of the cam slot of the first jaw member includes moving the pin a first distance, and moving the pin through the second portion of the cam slot of the first jaw member includes moving the pin a second distance, the first distance being different than the second distance.

16. The method according to claim 15, further including moving the pin through a first portion of a cam slot of the second jaw member, and moving the pin through a second portion of the cam slot of the second jaw member.

17. The method according to claim 16, wherein moving the pin through the first portion of the cam slot of the first jaw member occurs at the same time as moving the pin through the first portion of the cam slot of the second jaw member.

18. A jaw member of a surgical instrument, comprising:
an elongated portion defining a longitudinal axis; and
a cam slot disposed proximally of the elongated portion and configured to slidingly receive a cam pin, the cam slot defining a first portion having a first slope relative to the longitudinal axis and a second portion having a second slope relative to the longitudinal axis, the second slope being different than the first slope,
wherein distal movement of the cam pin along the first portion of the cam slot causes initial pivotal movement of the jaw member about a pivot pin, and distal movement of the cam pin along the second portion of the cam slot causes additional pivotal movement of the jaw member about the pivot pin.

19. The jaw member according to claim 18, wherein the first portion of the cam slot defines a first distance, and the second portion of the cam slot defines a second distance, the first distance being smaller than the second distance.

20. The jaw member according to claim 19, wherein the second slope of the cam slot defines a steeper angle relative to the longitudinal axis than the first slope of the cam slot.

21. The jaw member according to claim 18, wherein the second slope of the cam slot defines a steeper angle relative to the longitudinal axis than the first slope of the cam slot.
